(19) United States
(12) Patent Application Publication
(10) Pub. No.: US 2012/0035647 A1
(43) Pub. Date: Feb. 9, 2012

(54) BODY LUMEN FILTERS WITH LARGE SURFACE AREA ANCHORS

(76) Inventor: Rainer Bregulla, Balingen (DE)

(21) Appl. No.: 13/133,114

(22) PCT Filed: Dec. 16, 2009
(86) PCT No.: PCT/EP2009/009049

§ 371 Date: Sep. 22, 2011

Related U.S. Application Data
(60) Provisional application No. 61/138,406, filed on Dec. 17, 2008.

(51) Int. Cl. A61F 2/01

(52) U.S. Cl. 606/200

(57) ABSTRACT

As described herein, a body lumen filter (100) is provided that includes a body configured to move between a pre-deployed state and a deployed state. In the deployed state, the body has filtering openings (150) defined therein. The body lumen filter also includes at least one anchor (140) coupled to the body, the anchor including a base and a bulbed (170) portion, having a major cross-sectional dimension that is larger than a major cross-sectional dimension of the base.

### CROSS-REFERENCE TO RELATED APPLICATIONS

This Patent Application claims the benefit of and priority to U.S. Provisional Patent Application having Ser. No. 61/138,406, filed on Dec. 17, 2008, the disclosure of which is incorporated herein by reference in its entirety.

### BACKGROUND

1. Field of the Invention

The present disclosure relates generally to medical devices and to body lumen filters in particular, such as body lumen filters with large surface area anchors and methods for filtering a body lumen.

2. Background and Relevant Art

Surgical procedures, including both invasive as well as minimally-invasive procedures, save countless lives each year. However, the instrument and processes used during such procedures sometimes create additional challenges. For example, many minimally invasive procedures are performed using highly specialized surgical tools that are introduced to the procedure site by way of the patient's vasculature. In particular, a catheter is introduced into the vasculature by way of a small incision. The catheter is then advanced into proximity with the procedure site. Thereafter, the surgical tools are advanced to the procedure site through the catheter. With the surgical tools thus at the procedure site, the surgical tools are then manipulated from the outside of the body. Accordingly, a surgical procedure can be performed with only a small incision. While such an approach can reduce the invasiveness of performing a surgical procedure, this approach can cause additional challenges.

In particular, as the catheter and/or surgical devices are advanced through the vasculature, their passage can cause arterial plaques, clots, or other debris commonly referred to as emboli to become dislodged and move with the blood as it circulates through the vasculature. As the emboli move downstream, they can encounter plaque or other obstructions within the bloodstream to form new clots or obstructions in the bloodstream. Such obstructions can result in partial or complete blockage of vessels supplying blood and oxygen to critical organs, such as the heart, lungs and brain.

Accordingly, filter devices have been developed to capture the emboli at safe locations. Vena cava filters are devices that are implanted in the inferior vena cava, providing a mechanical barrier to undesirable particulates. The filters may be used to filter peripheral venous blood clots and other particulates, which if remaining in the blood stream can migrate in the pulmonary artery or one of its branches and cause harm.

Therefore, a body lumen filter with large surface area anchors and methods for filtering a body lumen may be useful.

### BRIEF SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

As described herein, a body lumen filter is provided that includes a body configured to move between a pre-deployed state and a deployed state. In the deployed state, the body has filtering openings defined therein. The body lumen filter also includes at least one anchor coupled to the body. The anchor may include a base and a bulbed portion. The bulbed portion may have a major cross-sectional dimension that is larger than a major cross-sectional dimension of the base.

In other examples, the body lumen filter can include a body configured to move between a pre-deployed state and a deployed state. The body lumen filter can also include a plurality of anchors secured to the body in which the anchors are configured to engage a body vessel at a deployment site with a force effective to maintain the body at the deployment site while having a surface area sufficient to prevent penetration of the anchors through an intima layer of the body vessel.

In other examples, a system is provided that can include a body lumen filter including a body having at least one anchor coupled to the body, the anchor including a base and a bulbed portion, the bulbed portion having cross-sectional dimensions that are larger than a largest cross-sectional dimension of the base. The system can also include a deployment device configured to move the body lumen filter between a pre-deployed state and a deployed state in which in the deployed state the body has filtering openings defined therein.

In another example, a method is described. The method may include longitudinally elongating the body of a body lumen filter such that the body lumen filter has a reduced dimension. The body lumen filter may be delivered to a desired deployment site within the body lumen. The body may be longitudinally reduced such that the body lumen filter has an enlarged dimension and the at least one anchor applies radial forces to an inner wall of the body lumen.

These and other features of the present invention will become more fully apparent from the following description and appended claims, or can be learned by the practice of the invention as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical examples and are not therefore to be considered to be limiting of the invention's scope, examples will be described and explained with additional specificity and detail through the use of the accompanying drawings in which:

FIG. 1A illustrates a body lumen filter in a deployed state according to one example;

FIG. 1B illustrates the anchor of the body lumen filter of FIG. 1A in more detail;

FIG. 1C illustrates a cross-sectional view of a bulbed portion of the anchor of FIG. 1B;

FIG. 2A illustrates a body lumen filter in a pre-deployed state being introduced to a body lumen by a deployment device according to one example;

FIG. 2B illustrates a body lumen filter in a deployed state and deployed in the body lumen according to one example;

FIG. 2C illustrates a removal step of the body lumen feature according to one example;

FIG. 3 illustrates an exemplary anchor configuration according to one example;

FIG. 4 illustrates an exemplary anchor configuration according to one example;

FIG. 5 illustrates an exemplary anchor configuration according to one example;

FIG. 6 illustrates an exemplary anchor configuration according to one example;

FIG. 7 illustrates an exemplary anchor configuration according to one example; and

FIG. 8 illustrates a body lumen filter according to one example.

### DETAILED DESCRIPTION

Devices and systems are provided herein for filtering a body lumen. By way of example only, a body lumen may include a blood vessel. Filtering may be performed by body lumen filters. For instance, embodiments of body lumen filters (e.g. including vena cava and/or other lumen filters), are described. Components of body lumen filters also are described. These components may include anchors and/or other components. In particular, the devices and systems provided herein include body lumen filters having a plurality of anchors with relatively large surface areas. The relatively large surface areas of the anchors can reduce the trauma associated with maintaining the body lumen filters at an intended location.

Some body lumen filters may be designed to capture and/or lyse particles of a particular size. Many body lumen filters may be generally tapered from a distal end toward a proximal end. For example, many body lumen filters may be generally cone shaped. Tapered body lumen filters may become misaligned within a body lumen. For instance, a tapered body lumen filter may be considered properly aligned within a body lumen or a longitudinal axis of the body lumen filter is generally aligned with a longitudinal axis of the body lumen at a deployment site. However, tapered body lumen filters, after delivery, may become improperly oriented such that the longitudinal axis of the implantable and filter is not aligned with a longitudinal axis of the body at the deployment site.

The body lumen filters and/or anchors described herein may be manufactured from any suitable material. For example, a body lumen filter and/or anchor may be, at least partially, formed from various materials including, but not limited to, nickel titanium and/or alloys thereof, stainless steel, cobalt chromium and/or alloys thereof, niobium tantalum and/or alloys thereof, other materials suitable for implantable stents, filters, or other implantable medical devices, and/or combinations thereof. Further, a body lumen filter and/or anchor may be, at least partially, formed of or include a radiopaque material and/or be coated with a radiopaque material to enhance visibility of the body lumen filter and/or the anchors.

These materials may include at least one beneficial agent incorporated into the material and/or coated over at least a portion of the material. The beneficial agents may be applied to body lumen filters that have been coated with a polymeric compound. Incorporation of the compound or drug into the polymeric coating of the body lumen filter can be carried out by dipping the polymer-coated body lumen filter into a solution containing the compound or drug for a sufficient period of time (such as, for example, five minutes) and then drying the coated body lumen filter, preferably by means of air drying for a sufficient period of time (such as, for example, 30 minutes). The polymer-coated body lumen filter containing the beneficial agent may then be delivered to a body vessel.

The pharmacologic agents that can be effective in preventing restenosis can be classified into the categories of anti-proliferative agents, anti-platelet agents, anti-inflammatory agents, anti-thrombotic agents, and thrombolytic agents. Anti-proliferative agents may include, for example, crystalline rapamycin. These classes can be further sub-divided. For example, anti-proliferative agents can be anti-mitotic. Anti-mitotic agents inhibit or affect cell division, whereby processes normally involved in cell division do not take place. One sub-class of anti-mitotic agents includes vinca alkaloids. Representative examples of vinca alkaloids include, but are not limited to, vincristine, paclitaxel, etoposide, nocodazole, indirubin, and anthracycline derivatives, such as, for example, daunorubicin, daunomycin, and plicamycin. Other sub-classes of anti-mitotic agents include anti-mitotic alkylating agents, such as, for example, tauromustine, bofumustine, and fotemustine, and anti-mitotic metabolites, such as, for example, methotrexate, fluorouracil, 5-bromodeoxyuridine, 6-azacytidine, and cytarabine. Anti-mitotic alkylating agents affect cell division by covalently modifying DNA, RNA, or proteins, thereby inhibiting DNA replication, RNA transcription, RNA translation, protein synthesis, or combinations of the foregoing.

Anti-platelet agents are therapeutic entities that act by (1) inhibiting adhesion of platelets to a surface, typically a thrombogenic surface, (2) inhibiting aggregation of platelets, (3) inhibiting activation of platelets, or (4) combinations of the foregoing. Activation of platelets is a process whereby platelets are converted from a quiescent, resting state to one in which platelets undergo a number of morphologic changes induced by contact with a thrombogenic surface. These changes include changes in the shape of the platelets, accompanied by the formation of pseudopods, binding to membrane receptors, and secretion of small molecules and proteins, such as, for example, ADP and platelet factor 4. Anti-platelet agents that act as inhibitors of adhesion of platelets include, but are not limited to, eptifibatide, tirofiban, RGD (Arg-Gly-Asp)-based peptides that inhibit binding to gpIIbIIIa or αvβ3, antibodies that block binding to gpIIaIIIb or αvβ3, anti-P-selectin antibodies, anti-E-selectin antibodies, compounds that block P-selectin or E-selectin binding to their respective ligands, saratin, and anti-von Willebrand factor antibodies. Agents that inhibit ADP-mediated platelet aggregation include, but are not limited to, disagregin and cilostazol.

Anti-inflammatory agents can also be used. Examples of these include, but are not limited to, prednisone, dexamethasone, hydrocortisone, estradiol, fluticasone, clobetasol, and non-steroidal anti-inflammatories, such as, for example, acetaminophen, ibuprofen, naproxen, and sulindac. Other examples of these agents include those that inhibit binding of cytokines or chemokines to the cognate receptors to inhibit pro-inflammatory signals transduced by the cytokines or the chemokines. Representative examples of these agents include, but are not limited to, anti-IL1, anti-IL2, anti-IL3, anti-IL4, anti-IL8, anti-IL15, anti-IL18, anti-GM-CSF, and anti-TNF antibodies.

Anti-thrombotic agents include chemical and biological entities that can intervene at any stage in the coagulation pathway. Examples of specific entities include, but are not limited to, small molecules that inhibit the activity of factor Xa. In addition, heparinoid-type agents that can inhibit both FXa and thrombin, either directly or indirectly, such as, for example, heparin, heparin sulfate, low molecular weight heparins, such as, for example, the compound having the trademark Clivarin®, and synthetic oligosaccharides, such as, for example, the compound having the trademark Arixtra®. Also included are direct thrombin inhibitors, such as, for example, melagatran, ximelagatran, argatroban, inogatran, and peptidomimetics of binding site of the Phe-Pro-Arg fibrinogen substrate for thrombin. Another class of anti-thrombotic agents that can be delivered is factor VII/VIIa inhibitors, such as, for example, anti-factor VII/VIIa antibodies, rNAPc2, and tissue factor pathway inhibitor (TFPI).

Thrombolytic agents, which may be defined as agents that help degrade thrombi (clots), can also be used as adjunctive agents, because the action of lysing a clot helps to disperse platelets trapped within the fibrin matrix of a thrombus. Representative examples of thrombolytic agents include, but are not limited to, urokinase or recombinant urokinase, pro-urokinase or recombinant pro-urokinase, tissue plasminogen activator or its recombinant form, and streptokinase.

One or more immunosuppressant agents may be used. Immunosuppressant agents may include, but are not limited to, IMURAN® azathioprine sodium, brequinar sodium, SPANIDIN® gusperimus trihydrochloride (also known as deoxyspergualin), mizoribine (also known as bredinin), CELLCEPT® mycophenolate mofetil, NEORAL® Cylosporin A (also marketed as different formulation of Cyclosporin A under the trademark SANDIMMUNE®), PROGRAF® tacrolimus (also known as FK-506), sirolimus and RAPAMUNE®, leflunomide (also known as HWA-486), glucocorticoids, such as prednisolone and its derivatives, antibody therapies such as orthoclone (OKT3) and Zenapax®, and antithymyocyte globulins, such as thymoglobulins. In addition, a crystalline rapamycin analog, A-94507, SDZ RAD (a.k.a. Everolimus), and/or other immunosuppressants.

Many body lumen filters may include hooks and/or other anchoring devices that pierce the inner wall of the body lumen to prevent filter migration. In some cases, piercing the inner wall of the body lumen may not be desirable. For instance, where the body lumen is already weakened. Body lumen filters that do not include hooks and/or other anchoring devices that pierce the inner wall of the body lumen may be subject to filter migration.

Thus, embodiments of the description relating to a body lumen filter with anchors having relatively large surface areas and methods for filtering a body lumen may be useful for facilitating filtering of a body lumen.

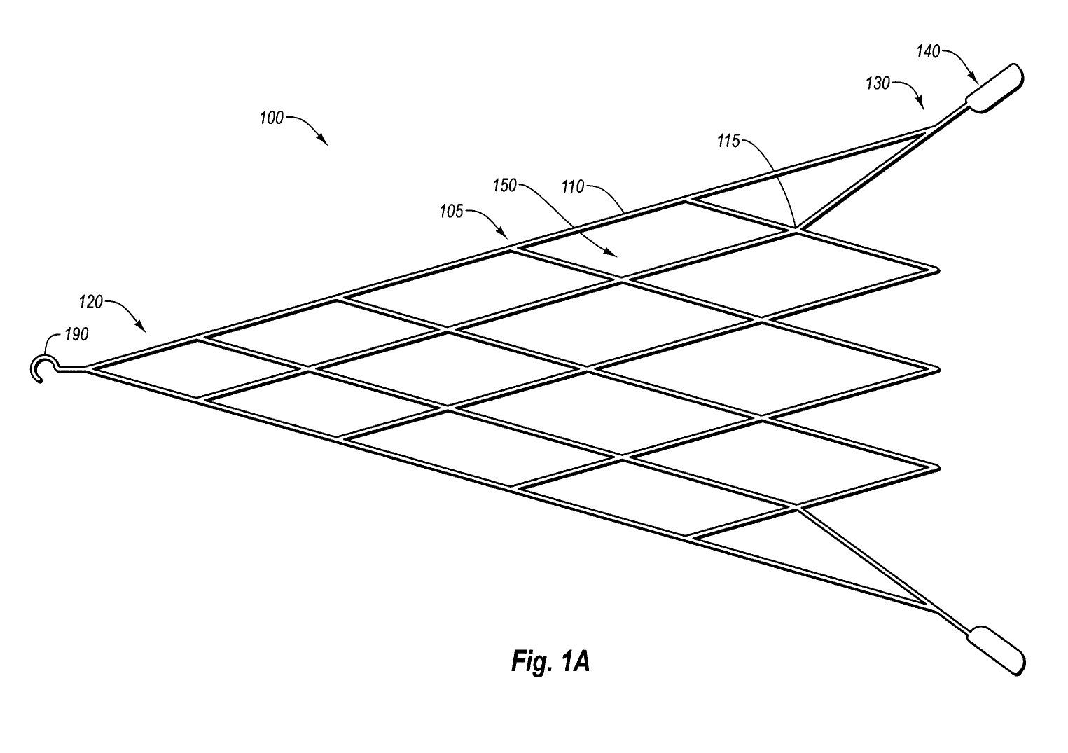

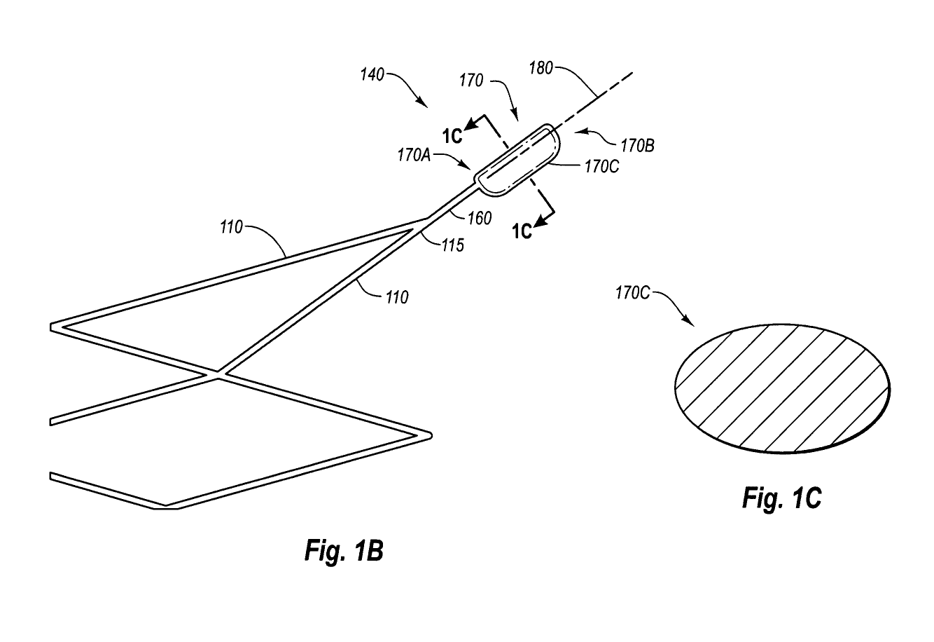

FIG. 1A illustrates a body lumen filter 100 for filtering a body lumen, such as a blood vessel. As illustrated in FIG. 1A, the body lumen filter 100 includes a body 105 having a plurality of expandable struts 110. The expandable struts 110 are interconnected at junctions 115 in such a manner as to allow the body lumen filter 100 to be moved from a pre-deployed configuration to a deployed configuration. In at least one example, the pre-deployed configuration can be a relatively collapsed configuration. In such examples, the body lumen filter can be moved from the pre-deployed configuration to the deployed configuration by expanding at least a portion of the body lumen filter 100.

In the illustrated example, the body lumen filter 100 can be expanded by providing relative separation between at least some of the expandable struts 110 and/or junctions 115. The expandable struts 110 can be expanded in any suitable manner. In at least one example, the expandable struts 110 can be mechanically expanded by an expansion member, such as a balloon or other expansion member. In other examples the expandable struts 110 can be formed of a material that can resiliently move from a pre-deployed state to a deployed state due to the resilient nature of the material.

In FIG. 1A the body lumen filter 100 is shown in a deployed state. The body lumen filter 100 includes a first end 120 and a second end 130. At least one anchor 140 is coupled to at least one of the first end 120 or the second end 130. In the deployed state, filter openings 150 are defined between one or more of the expandable struts 110 and the junctions 115. Further, in the example shown the first end 120 is narrower than the second end 130 such that the body lumen filter 100 has a generally tapered shape.

In other examples, the body lumen filter 100 may have different shapes, such as an hourglass shape, a generally parabolic shape, other shapes, or combinations thereof. Additionally, in the illustrated example the body 105 is formed of expandable struts 110. In other examples, the body 105 can be formed of other components, such as helically wound wires, twisted wires, other elements or combinations thereof that form a plurality of filter openings 150.

In the illustrated example, anchors 140 are secured to the expandable struts 110 on at least the second end 130. The anchors 140 may have relatively high-surface areas as compared to a conventional anchor in which the anchor is achieved by bending an end of an expandable strut or other elongate portion of the body lumen filter away from a longitudinal axis of the body lumen filter 100. The relatively high surface area of the anchor 140 can allow the anchor 140 to secure the body lumen filter 100 at a deployed position. The relatively high surface area may decrease the potential that the anchor 140 will pierce through the inner surface of the body lumen (i.e. an intima of a blood vessel) in which the body lumen filter is deployed. One exemplary configuration of an anchor 140 and an exemplary configuration of an associated body lumen filter will now be discussed in more detail.

As illustrated in FIG. 1B, two of the expandable struts 110 meet at the junction 115. The anchor 140 is shown extending away from a longitudinal axis of the body lumen filter and away from the junction 115. In particular, the anchor 140 may include a base 160 secured to the junction 115. Alternatively, the base 160 may be a portion of the body 105. For instance, the anchor 140 may be connected to a strut 110, a junction 115, or other portions of the body 105. The anchor 140 further includes a bulbed portion 170 that may be secured to the base 160.

The bulbed portion 170 may include a first end 170A and a second end 170B. The anchor 140 can be described with reference to a primary axis 180 that can extend generally along a centerline of the anchor 140 and/or base 160. The primary axis 180 can then extend along a portion of the bulbed portion 170 between the first end 170A and the second end 170B. The primary axis 180 is referenced for ease of discussion in describing a relative orientation and location of the bulbed portion 170 relative to the base 160. In at least one example, the bulbed portion 170 can be offset relative to the primary axis 180 such that a majority of the bulbed portion 170 is oriented away from the primary axis 180.

In the illustrated example, the bulbed portion 170 can have a three-dimensional shape in which at least part of the bulbed portion 170 has cross-sectional dimensions that are larger than a largest cross-sectional dimension of the base 160 or a portion of the body 105 to which the bulbed portion 170 may be connected (such as a strut 110, a junction 115, or other component). Cross-sectional dimensions can refer to the largest dimension of a cross-sectional portion when a cross section is taken normally relative to a center axis or line of the corresponding component. Such a center line can follow any path, including straight, curved, arced, other paths, or combinations thereof. In the illustrated example, the primary axis 180 can be co-linear with a center line of the base 160 such that cross sections of the base 160 are taken normally relative to the primary axis 180.

In at least one example, the anchor 140 has as a three-dimensional elliptical shape, which can be referred to as an ovoid shape. Such a configuration may result in at least part of the bulbed portion 170 having a cross-sectional dimension that is larger than a corresponding dimension of the base 160 or a portion of the body 105 to which the bulbed portion 170 may be connected.

In the illustrated example, the second end 170B of the bulbed portion 170 can be shaped such that the second end 170B transitions smoothly to an intermediate portion 170C. Further, the first end 170A can be shaped to form a smooth transition between the base 160 and the central portion 170C. In other examples, the transition can be step-wise, irregular, any other type of transition, or combinations of the same. Further, the first end 170A and/or second end 170B may be shaped similarly to the central portion 170C such that there is generally no transition. The central portion 170C in turn can have a generally smooth cross-sectional profile or can have an irregular cross-sectional shape as desired.

FIG. 1C illustrates a cross-sectional view of the central portion 170C of one exemplary bulbed portion 170 in more detail taken along section 1C-1C in FIG. 1B. In the illustrated example, the central portion 170C can have a generally elliptical shape. Other cross-sectional shapes can include round or rounded cross-sectional shapes, sharply transitioned areas, as well as any other shapes or combinations thereof. Further, it will be appreciated that the central bulbed portion 170C, as well as any other part of the bulbed portion 170 can have any shape desired to provide a relatively high-surface area for the anchor 140. Various shape configurations can allow one or more anchor 140 to secure the body lumen filter 100 at a deployed location while reducing the penetration of the anchor 140 through the inner layer of the body lumen in which it is deployed. One exemplary process for deploying the body lumen filter 100 and the anchors 140 in particular will be described below.

Referring now to both FIGS. 1B and 1C, the bulbed portion 170 can have a length of between about 1 mm and about 30 mm and a largest cross-sectional dimension of between about 0.10 mm and about 5 mm. The base 160 can have a largest cross-sectional dimension of between about 0.05 mm and about 1 mm. Accordingly, the anchor 140 can have a relatively large surface area. Further, an engagement surface area of the anchor 140, which can be defined as the surface area of anchor 140 that may contact an inner surface of a body lumen. For instance, the engagement surface area may include one half of total surface area of the bulbed portion 170 can be between about 0.10 mm² and about 150 mm². In another example, the engagement surface area may include the surface area of the hemisphere of the anchor 140 that may contact the inner surface of the body lumen.

The bulbed portion 170 may be shaped to conform to an inner surface of a body lumen. For example, the engaging surface may be generally rounded to approximate the curvature of the inner surface of the body lumen.

The body lumen filter 100 may be formed in any manner. In at least one example, the body 105 may be formed first, such as by etching, cutting, rolling, welding, or any combination of processes to form expandable struts 110, junctions 115, and/or bases 160. Thereafter, the bulbed portions 170 may be formed on and/or attached to the bases 160, junctions 115, struts 110, other portions of the base 105, or combinations thereof. The bulbed portions 170 may be formed in any manner, such as through deposition processes, soldering, molding, other operations, or combinations thereof. The body lumen filter 100 can further include an engagement feature 190 coupled to a first end 120 thereof.

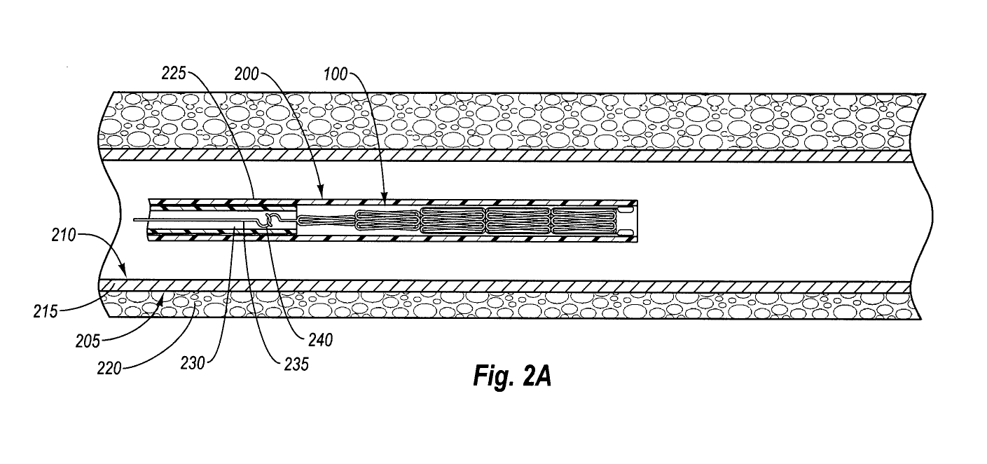

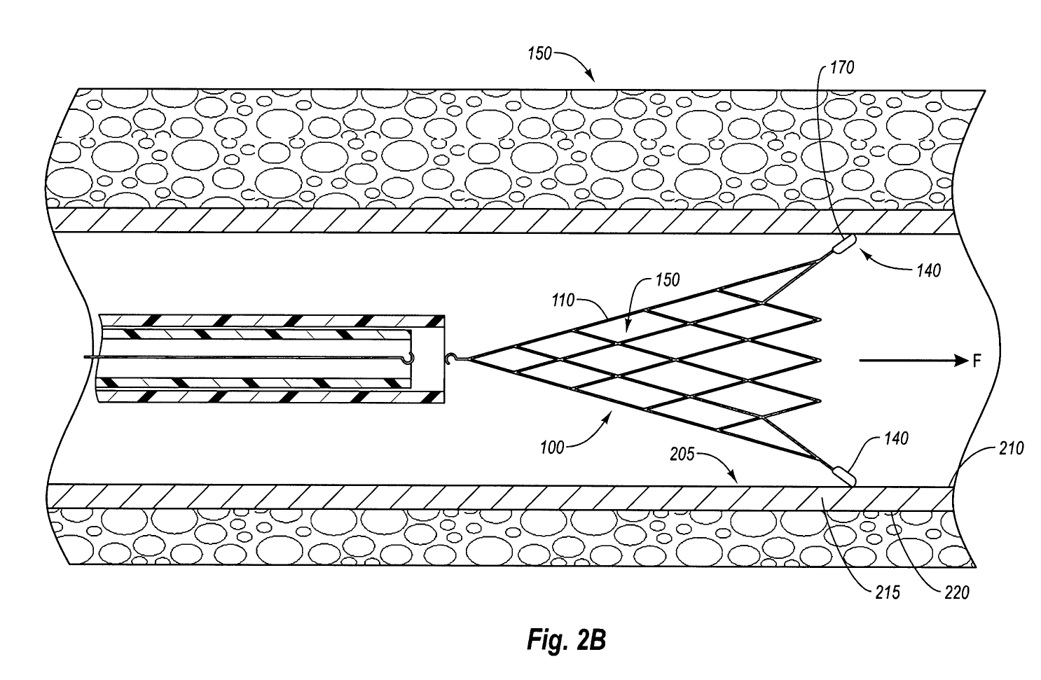

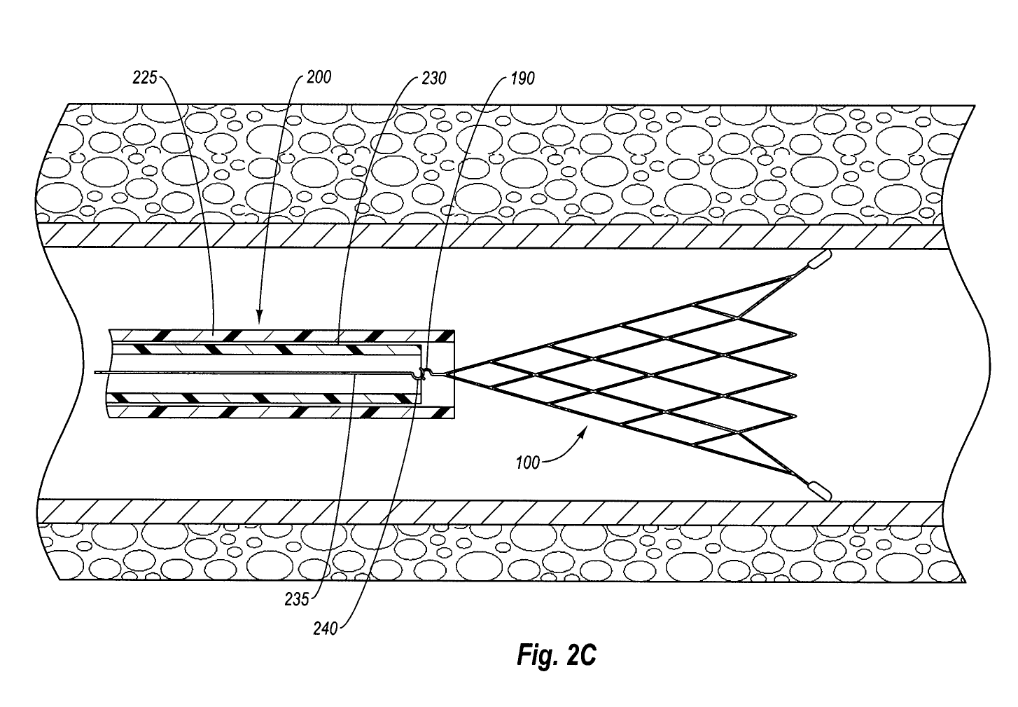

FIG. 2A illustrates the body lumen filter 100 in a pre-deployed configuration and located within a deployment device 200. The deployment device 200 is configured to deploy the filter 100 into a body lumen 205. The body lumen 205 may include an inner layer 210 (i.e. intima layer), a medial layer 215, an adventitial layer 220, or combinations thereof. The deployment device 200 can include a housing 225 and/or a delivery mechanism 230 that may be actuated from a proximally located handle (not shown). A retrieval device 235 may be positioned within the delivery mechanism 230 and may similarly be actuated from the proximally located handle. The retrieval device 235 can include a retrieval feature 240 configured to engage the engagement feature 190 on the body lumen filter 100.

As previously discussed, the body lumen filter 100 includes expandable struts 110 that are interconnected in such a manner as to allow the body lumen filter 100 to be moved from the pre-deployed state illustrated in FIG. 2A to a deployed state illustrated in FIG. 2B. To deploy the body lumen filter 100, the deployment device 200 is moved near a desired location within a body lumen 205 by using a catheter or other techniques. In one example, once the deployment device 200 is near the desired location, the delivery mechanism 230, such as a plunger or other moveable member disposed within the housing 225, may be advanced distally relative to the housing 225, thereby driving the body lumen filter 100 from the housing 225 toward the desired location. In another example, the deployment device 200 may be advanced to the desired location, the delivery mechanism 230 may be advanced distally to abut the body lumen filter 100, the housing 225 may be retracted to deploy the body lumen filter 100, or combinations thereof.

As the body lumen filter 100 is deployed by the deployment device 200, the body lumen filter 100 may move toward the deployed state. For instance, for a body lumen filter 100 formed from a shape memory material, moving the delivery mechanism 230 distally, moving the housing 225 proximally, or a combination of such movements, may release the body lumen filter 100 from within the housing 225 to transition to the deployed state of FIG. 2B.

A resilient body lumen filter 100 is illustrated. Embodiments of the body lumen filter body 105 and/or anchor 140 can include a material made from any of a variety of known suitable materials, such as a shape memory material (SMM). For example, the SMM can be shaped in a manner that allows for restriction to induce a substantially reduced, generally linear orientation while within the housing 225, but can automatically return to the memory shape of the body lumen filter 100 once extended from the deployment device 200. SMMs have a shape memory effect in which they can be made to remember a particular shape. Once a shape has been remembered, the SMM can be bent out of shape or deformed and then returned to its original shape by unloading from strain and/or heating. Typically, SMMs can be shape memory alloys (SMA) comprised of metal alloys, or shape memory plastics (SMP) comprised of polymers. The materials can also be referred to as being superelastic.

Usually, an SMA can have any non-characteristic initial shape that can then be configured into a memory shape by heating the SMA and conforming the SMA into the desired memory shape. After the SMA is cooled, the desired memory shape can be retained. This allows for the SMA to be bent, straightened, compacted, and placed into various contortions by the application of requisite forces; however, after the forces are released, the SMA can be capable of returning to the memory shape. The main types of SMAs are as follows: copper-zinc-aluminium; copper-aluminium-nickel; nickel-titanium (NiTi) alloys known as nitinol; and cobalt-chromium-nickel alloys nickel-titanium platinum; nickel-titanium palladium or cobalt-chromium-nickel-molybdenum alloys known as elgiloy alloys. The temperatures at which the SMA changes its crystallographic structure are characteristic of the alloy, and can be tuned by varying the elemental ratios or by the conditions of manufacture.

For example, the material of a body lumen filter 100 can be of a NiTi alloy that forms superelastic nitinol. In the present case, nitinol materials can be trained to remember a certain shape, straightened in a shaft, catheter, or other tube, and then released from the catheter or tube to return to its trained shape. Also, additional materials can be added to the nitinol depending on the desired characteristic. The alloy can be utilized having linear elastic properties or non-linear elastic properties.

An SMP is a shape-shifting plastic that can be fashioned into a body lumen filter 100 in accordance with the present invention. Also, it can be beneficial to include at least one layer of an SMA and at least one layer of an SMP to form a multilayered body;

however, any appropriate combination of materials can be used to form a multilayered body lumen filter 100. When an SMP encounters a temperature above the lowest melting point of the individual polymers, the blend makes a transition to a rubbery state. The elastic modulus can change more than two orders of magnitude across the transition temperature (Ttr). As such, an SMP can formed into a desired shape of a body lumen filter 100 by heating it above the Ttr, fixing the SMP into the new shape, and cooling the material below Ttr. The SMP can then be arranged into a temporary shape by force, and then resume the memory shape once the force has been applied. Examples of SMPs include, but are not limited to, biodegradable polymers, such as oligo(ε-caprolactone)diol, oligo(ρ-dioxanone)diol, and non-biodegradable polymers such as, polynorborene, polyisoprene, styrene butadiene, polyurethane-based materials, vinyl acetate-polyester-based compounds, and others yet to be determined. As such, any SMP can be used in accordance with the present invention.

A body lumen filter body 105 having at least one layer made of a SMM or suitable superelastic material and other suitable layers can be compressed or restrained in its delivery configuration within a delivery device using a sheath or similar restraint, and then deployed to its desired configuration at a deployment site by removal of the restraint as is known in the art. A body lumen filter body 105 made of a thermally-sensitive material can be deployed by exposure of the body lumen filter 100 to a sufficient temperature to facilitate expansion as is known in the art. It will be appreciated that the body lumen filter 100 can be mechanically expanded, such as by a balloon or other expanding device.

Continuing with the example illustrated in FIG. 2B, as the body lumen filter 100 is moved toward the expanded state, the expandable struts 110 can be displaced to provide the filter openings 150. The filter openings 150 can be sized to prevent particulates, such as at least one embolus, from passing through the body lumen filter 100 and/or to lyse particulates of greater than a desired size. While an embolus is trapped against body lumen filter 100, blood may continue to flow over the embolus. The flow of blood over the embolus can dissolve the embolus through the body's lysing process. Additionally, the body lumen filter 100 may be coated with a beneficial agent that may facilitate lysing of the embolus.

The blood flow F exerts a fluid force on the body lumen filter 100 that would tend to move the body lumen filter 100 in the direction of the blood flow F. The anchors 140 may resist this force to generally maintain the body lumen filter 100 in or near an intended deployment location. In particular, frictional, compressive, and/or other forces between the body lumen filter 100 and the body lumen 205 may generally maintain the body lumen filter 100 at or near the intended deployment location, as will now be described in more detail below.

As the body lumen filter 100 is moved toward the deployed state, the anchors 140 may be moved into contact with the intima layer 210 of the body lumen 205. In the deployed state, opposing anchors 140 can be separated by a distance that is slightly larger than the diameter of the body lumen 205 before the body lumen filter 100 is deployed. As a result, a tensile force can urge or press the bulbed portion 170 of one or more of the anchors 140 into contact with the intima layer 210.

As the bulbed portions 170 are urged into contact with the intima layer 210, the intima layer 210 can deform slightly to begin to conform to the shape of the bulbed portion 170, which can result in compressive forces between the bulbed portion 170 and the body lumen 205.

Further, this deformation can increase contact between the arcuate anchor 170 and the intima layer 210. Frictional forces between two objects that are in contact typically depend on the normally applied force and the coefficient of friction between the two objects. The normally applied force depends on the area of contact and the pressure applied to that area. The coefficient of friction as well as the normal force necessary to maintain the body lumen filter 100 positioned in body lumen 205 may be relatively constant. Accordingly, increasing the surface area over which the arcuate anchor 170 applies the normal force can reduce the pressure the arcuate anchor 170 applies to the intima layer 210 of the body lumen 205. Decreasing the pressure applied to the body lumen 205, in turn, can reduce the possibility that the arcuate anchors 170 will pierce the intima layer 210.

Accordingly, the relatively large surface area of the anchors 140 can help maintain the body lumen filter 100 at or near a desired deployment location in the body lumen 205. Further, the relatively large surface area of the anchors 140 can reduce the likelihood that the anchors 140 will penetrate through the intima layer 210 and into the medial layer 215 and/or the adventitial layer 220. Reducing penetration into the medial layer 215 may in turn reduce endothelial growth while and/or after the body lumen filter 100 is deployed.

At some point, it may be desirable to retrieve the body lumen filter 100. FIG. 2C illustrates a step for retrieving the body lumen filter 100. As illustrated in FIG. 2C, retrieving the body lumen filter 100 can include positioning the deployment device 200 such that the housing 225 is positioned in proximity to the body lumen filter 100. Thereafter, the retrieval device 235 can be moved into engagement with the engagement feature 190 on the body lumen filter 100. In particular, the retrieval feature 240 can be advanced distally beyond the housing 225 and into engagement with engagement feature 190 or some other portion of the body lumen filter. Thereafter, the retrieval device 235 can be proximally relative to the housing 225 and/or the deployment mechanism 230 to thereby draw the body lumen filter 200 into the deployment device 200. Once the body lumen filter 200 is located within the deployment device 200, the deployment device 200 can be removed to thereby complete retrieval of the body lumen filter 100.

Referring briefly again to FIG. 1B, the first end 170A and the second end 170B can transition smoothly to the central portion 170C. Further, the central portion 170C can include a generally elliptical cross-sectional shape. Such a configuration can provide a bulbed portion 170 that can have arcuate or smooth edges. Referring again to FIG. 2B, relatively smooth edges can distribute the compressive forces discussed above in a generally even manner across the bulbed portion 170 to thereby reduce the likelihood that the anchor 140 will pierce the intima layer 210. Reducing the likelihood of penetration of the anchor 140 through the intima layer 210 can in turn reduce endothelial growth, which can reduce trauma associated with retrieval of the body lumen filter 100, as described above.

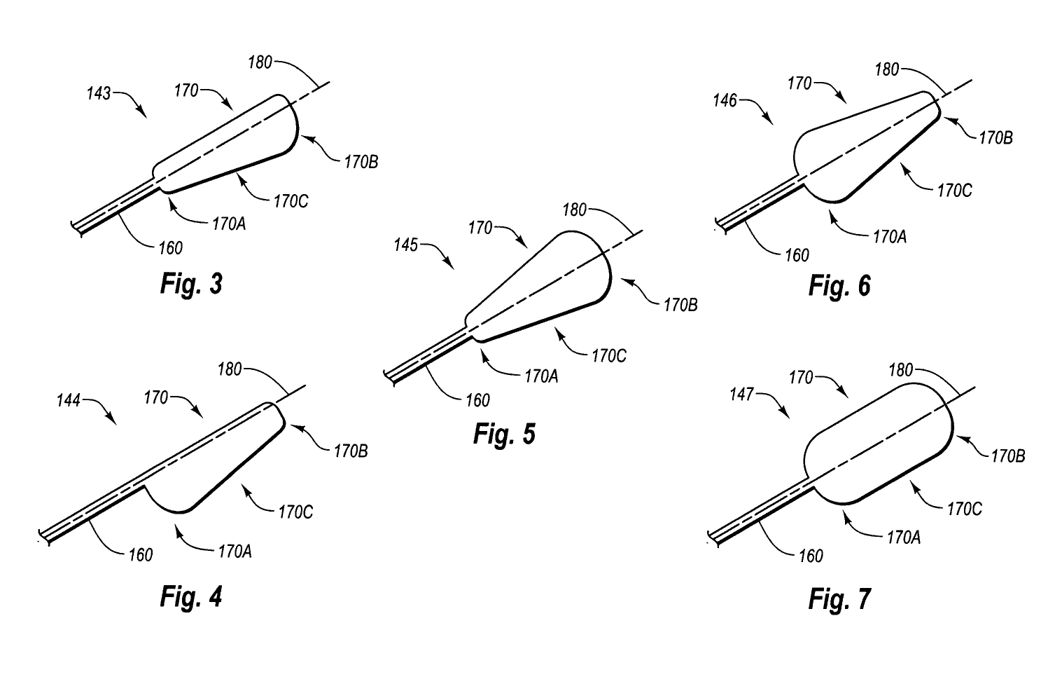

Anchors can be provided having any number of shapes and configurations that have relatively high-surface area. These shapes can include shapes having rounded edges and/or non-rounded edges. Additional configurations of anchors with rounded configurations are shown in FIGS. 3-7.

FIG. 3 illustrates an anchor 143 having a bulbed portion 170. As illustrated in FIG.

3, the bulbed portion 170 is offset relative to the primary axis 180. Further, the anchor 143 illustrated in FIG. 3 has a second end 170B that has a larger cross-sectional area than an intermediate portion 170C. The intermediate portion 170C in turn has a larger cross-sectional area than a first end 170A of the bulbed portion 170.

In another example illustrated in FIG. 4, an anchor 144 can include a bulbed portion 170 having a first end 170A with a larger cross-sectional area than an intermediate portion 170C. The intermediate portion 170C can in turn have a larger cross-sectional area than a second end 170B of the bulbed portion 170.

FIG. 5 illustrates an anchor 145 that includes a bulbed portion 170 having second end 170B that has a larger cross-sectional area than an intermediate portion 170C. The intermediate portion 170C in turn has a larger cross-sectional area than a first end 170A of the bulbed portion 170. Further, a primary axis 180 can pass more centrally through the bulbed portion 170.

A similar configuration illustrated in FIG. 6, in which an anchor 146 includes a bulbed portion 170 having a primary axis 180 passing more centrally through the bulbed portion 170. In the example illustrated in FIG. 6, a first end 170A of the bulbed portion has a larger cross-sectional area than an intermediate portion 170C. The intermediate portion 170C can in turn have a larger cross-sectional area than a second end 170B of the anchor 146.

A further anchor 147 is illustrated in FIG. 7, in which an intermediate portion 170C of the anchor 147 has a larger cross-sectional area than both a first end 170A and a second end 170B. In the illustrated example, a primary axis 180 passes substantially centrally through the bulbed portion 170.

In the examples illustrated above, a primary axis has been described which is generally aligned or parallel to a base portion of the anchor. In other examples, the primary axis and the base portion can be oriented at an angle relative to each other. Such orientations can be combined with any of the configurations of the bulbed portions described above, as well as any other configuration of an anchor having large surface area.

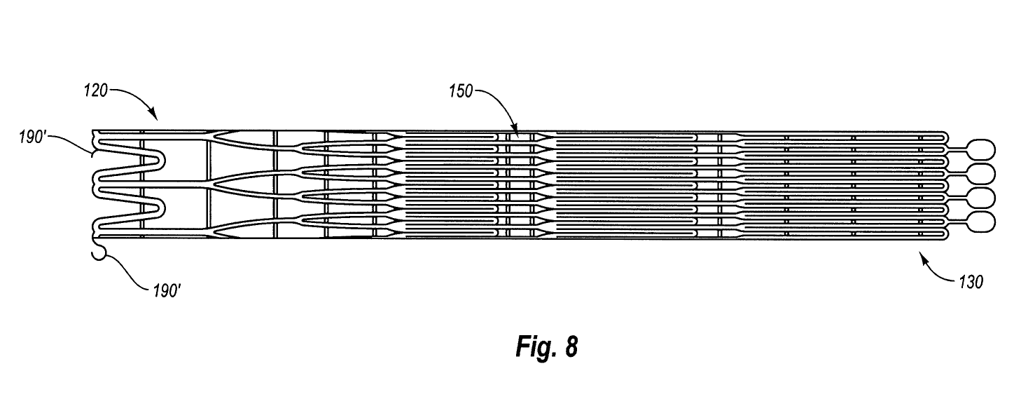

FIG. 8 illustrates a body lumen filter 100′ according to one example. As illustrated in FIG. 8, the number of filter openings 150, or cells, may increase toward the second end 130 of the body lumen filter 100′. A plurality of retrieval features 190′ may be positioned on various sides of the first end 120. As illustrated, the retrieval features 190′ may be positioned on opposing sides. While two retrieval features 190′ are shown, the number of these retrieval features 190′ could also be three, four, or more. The retrieval features 190′ may incorporate radiopaque materials to facilitate retrieval of the filter 100′. In some embodiments, the retrieval features 190′ may be located anywhere along the filter 100′.

The present invention can be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A body lumen filter, comprising:
a body configured to move between a pre-deployed state and a deployed state, wherein in the deployed state the body has filtering openings defined therein; and
at least one anchor coupled to the body, the anchor including a base and a bulbed portion, the bulbed portion having a major cross-sectional dimension that is larger than a major cross-sectional dimension of the base.

2. The body lumen filter of claim 1, wherein the body includes a first end and a second end and wherein the anchor is associated with the second end.

3. The body lumen filter of claim 2, wherein the second end is wider than the first end when the body is in a deployed state.

4. The body lumen filter of claim 2, wherein the anchor has a generally ovoid shape.

5. The body lumen filter of claim 1, wherein the bulbed portion includes a first end, a second end, and an intermediate portion and wherein each of the first end, the intermediate portion and the second end have a major cross-sectional dimension that is larger than a cross section of the base.

6. The body lumen filter of claim 5, wherein the intermediate portion has an elliptical cross-sectional shape.

7. The body lumen filter of claim 6, wherein the first end is rounded.

8. The body lumen filter of claim 5, wherein the major cross-sectional dimension of the intermediate portion is larger than the major cross-sectional dimension of the second end.

9. The body lumen filter of claim 8, wherein the major cross-sectional dimension of the intermediate portion is larger than the major cross-sectional dimension of the first end.

10. The body lumen filter of claim 1, wherein a primary axis is defined along a length of the base and wherein the bulbed anchor is at least partially offset relative to the primary axis.

11. The body lumen filter of claim 1, wherein a primary axis is defined along a length of the base and wherein the bulbed anchor is centered relative to the primary axis.

12. The body lumen filter of claim 1, wherein the body includes a plurality of struts and junctions joining adjacent struts.

13. A body lumen filter, comprising:
a body configured to move between a pre-deployed state and a deployed state,
a plurality of anchors secured to the body, the anchors being configured to engage a body vessel at a deployment site with a force effective to maintain the body at the deployment site while having a surface area sufficient to prevent penetration of the anchors through an inner surface of the body lumen while the body is in the deployed state at the deployment site.

14. The body lumen filter of claim 13, wherein the anchors have an engagement surface area of between about 0.10 mm² and about 150 mm².

15. The body lumen filter of claim 13, wherein each anchor includes a base coupled to the body and a bulbed portion coupled to the base.

16. The body lumen filter of claim 15, wherein the bulbed portion has a generally ovoid shape.

17. A system comprising
a body lumen filter including a body having at least one anchor coupled to the body, the anchor including a base and a bulbed portion, the bulbed portion having cross-sectional dimensions that are larger than a largest cross-sectional dimension of the base;
a deployment device configured to move the body lumen filter between a pre-deployed state and a deployed state, wherein in the deployed state the body has filtering openings defined therein.

18. The system of claim 17, wherein the bulbed portion has a generally ovoid shape.

19. The system of claim 17, wherein the bulbed portion has an engagement surface area of between about 0.10 mm² and about 150 mm².

20. The system of claim 17, wherein the body is formed from a resilient material.

21. A method for filtering a body lumen, the method comprising:
providing a body lumen filter comprising:
a body configured to move between a pre-deployed state and a deployed state, wherein in the deployed state the body has filtering openings defined therein; and
at least one anchor coupled to the body, the anchor including a bulbed portion, the bulbed portion having a major cross-sectional dimension that is larger than a major cross-sectional dimension of the body;
longitudinally elongating the body such that the body lumen filter has a reduced dimension;
delivering the body lumen filter to a desired deployment site within the body lumen; and
longitudinally reducing the body such that the body lumen filter has an enlarged dimension and the at least one anchor applies radial forces to an inner wall of the body lumen.

22. The method of claim 21, further comprising after longitudinally reducing the body, longitudinally elongating the body such that the body lumen filter has a reduced dimension and removing the body lumen filter from the desired deployment site within the body lumen.
